SEQUENCE LISTING (15 sequences)

SEQ ID NO: 1 - protein, 291 aa, Artificial Sequence
  human SGCG
MVREQYTTATEGICIERPENQYVYKIGIYGWRKRCLYLFVLLLLIILVVNLALTIWILKVMWFSPAGMGHLCVTKDGLRLEGESEFLFPLYAKEIHSRVDSSLLLQSTQNVTVNARNSEGEVTGRLKVGPKMVEVQNQQFQINSNDGKPLFTVDEKEVVVGTDKLRVTGPEGALFEHSVETPLVRADPFQDLRLESPTRSLSMDAPRGVHIQAHAGKIEALSQMDILFHSSDGMLVLDAETVCLPKLVQGTWGPSGSSQSLYEICVCPDGKLYLSVAGVSTTCQEHSHICL

SEQ ID NO: 2 - protein, 291 aa, Artificial Sequence
  human SGCG variant
MVREQYTTATEGICIERPENQYVYKIGIYGWRKRCLYLFVLLLLIILVVNLALTIWILKVMWFSPAGMGHLCVTKDGLRLEGESEFLFPLYAKEIHSRVDSSLLLQSTQNVTVNARNSEGEVTGRLKVGPKMVEVQNQQFQINSNDGKPLFTVDEKEVVVGTDKLRVTGPEGALFEHSVETPLVRADPFQDLRLESPTRSLSMDAPRGVHIQAHAGKIEALSQMDILFHSSDGMLVLDAETVCLPKLVQGTWGPSGSSQSLYEICVCPDGKLYLSVAGVSTTCQEHNHICL

SEQ ID NO: 3 - DNA, 876 bp, Artificial Sequence
  Synthetic Polynucleotide
  misc_feature: human SGCG
atggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgctgctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgctcgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacagccacatctgcctctaa

SEQ ID NO: 4 - DNA, 714 bp, Artificial Sequence
  Synthetic Polynucleotide
  misc_feature: tMCK
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccctccctggggacagcccctcctggctagtcacaccctgtaggctcctctatataacccaggggcacaggggctgcccccgggtca

SEQ ID NO: 5 - DNA, 2833 bp, Artificial Sequence
  Synthetic Polynucleotide
  misc_feature: K7 promoter-polyA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccctccctggggacagcccctcctggctagtcacaccctgtaggctcctctatataacccaggggcacaggggctgcccccgggtcacaccggtgctagcgttaacgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttttggttgggataaggctggattattctgagtccaagctaggcccttttgctaatcttgttcatacctcttatcttcctcccacagctcctgggcaacgtgctggtctctgtgctggcccatcactttggcaaagaattcgccaccatggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgctgctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgctcgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacagccacatctgcctctaagggcgaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 6 - DNA, 3149 bp, Artificial Sequence
  Synthetic Polynucleotide
  misc_feature: K7 ITR-ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctacgcgtgcggccgcggatccgtcgacccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggccctccctggggacagcccctcctggctagtcacaccctgtaggctcctctatataacccaggggcacaggggctgcccccgggtcacaccggtgctagcgttaacgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttttggttgggataaggctggattattctgagtccaagctaggcccttttgctaatcttgttcatacctcttatcttcctcccacagctcctgggcaacgtgctggtctctgtgctggcccatcactttggcaaagaattcgccaccatggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgctgctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgctcgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacagccacatctgcctctaagggcgaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 7 - DNA, 18 bp, Artificial Sequence
  Synthetic Polynucleotide
  misc_feature: PolyA HBB2 FWD
ccaggcgaggagaaacca

SEQ ID NO: 8 - DNA, 25 bp, Artificial Sequence
  Synthetic Polynucleotide
  misc_feature: PolyA HBB2 REV
cttgactccactcagttctcttgct

SEQ ID NO: 9 - DNA, 29 bp, Artificial Sequence
  Synthetic Polynucleotide
  misc_feature: PolyA HBB2 Probe
ctcgccgtaaaacatggaaggaacacttc

SEQ ID NO: 10 - DNA, 20 bp, Artificial Sequence
  Synthetic Polynucleotide
  misc_feature: P0 FWD
ctccaagcagatgcagcaga

SEQ ID NO: 11 - DNA, 20 bp, Artificial Sequence
  Synthetic Polynucleotide
  misc_feature: P0 REV
atagccttgcgcatcatggt

SEQ ID NO: 12 - DNA, 22 bp, Artificial Sequence
  Synthetic Polynucleotide
  misc_feature: P0 Probe
ccgtggtgctgatgggcaagaa

SEQ ID NO: 13 - DNA, 1052 bp, Artificial Sequence
  Synthetic Polynucleotide
  misc_feature: desmin promoter
tacccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 14 - DNA, 450 bp, Artificial Sequence
  Synthetic Polynucleotide
  misc_feature: CK8 promoter
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 15 - DNA, 792 bp, Artificial Sequence
  Synthetic Polynucleotide
  misc_feature: MHCK7 promoter
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

(19) United States
(12) Patent Application Publication
(10) Pub. No.: US 2023/0256117 A1
(43) Pub. Date: Aug. 17, 2023

(54) GENE THERAPY EXPRESSION SYSTEM ALLOWING AN ADEQUATE EXPRESSION IN THE MUSCLES AND IN THE HEART OF SGCG

(71) Applicants: GENETHON, Evry-Courcouronnes (FR); INSERM (INSTITUT NATIONAL DE LA SANTE ET DE LA RECHERCHE MEDICALE), Paris (FR); UNIVERSITE D’EVRY-VAL-D’ESSONNE, Evry-Courcouronnes (FR)

(72) Inventors: Isabelle RICHARD, Corbeil Essonnes (FR); Jerome POUPIOT, Mennecy (FR)

(21) Appl. No.: 18/001,648

(22) PCT Filed: Jun. 18, 2021
(86) PCT No.: PCT/EP2021/066626

§ 371 Date: Dec. 13, 2022

(30) Foreign Application Priority Data
Jun. 19, 2020 (EP) 20315308.5

(51) Int. Cl. A61K 48/00

(52) U.S. Cl. CPC A61K 48/0058

(57) ABSTRACT

The present invention concerns an expression system for systemic administration comprising a sequence encoding gamma-sarcoglycan (SGCG) placed under the control of a promoter allowing an adequate expression of SGCG in the skeletal muscles and in the heart, and its use for the treatment of Limb-Girdle Muscular Dystrophy type C.

The present invention is based on the identification of the benefit of an adequate expression of SGCG (y-sarcoglycan) in the skeletal muscles and in the heart, advantageously a quantity of SGCG protein in the skeletal muscles superior or equal to that in the heart. It provides an expression system combining the transgene and a promoter sequence, which avoids an excessive production in the heart. It then offers a valuable and safe therapeutic tool for the treatment of Limb-Girdle Muscular Dystrophy type 2C (LGMD2C), newly named Limb girdle muscular dystrophy type R5 (LGMD R5). Such an expression profile is also of interest for the other sarcoglycans, i.e. alpha (α)-sarcoglycan (SGCA), beta (β)-sarcoglycan (SGCB) and delta (8)-sarcoglycan (SGCD).

### BACKGROUND OF THE INVENTION

The term sarcoglycanopathies (SGs) comprises four different rare diseases belonging to the larger group of the limb girdle muscular dystrophies (LGMDs): LGMD2C or γ-SG, LGMD2D or α-SG, LGMD2E or β-SG, and LGMD2F or δ-SG. Interestingly, the relative frequency of each form varies enormously between different geographical areas. For example, LGMD2F represents about 14% of SGs in Brazil while being extremely rare elsewhere (Moreira E.S. et al., J. Med. Genet. 2003; 40:E12) and LGMD2C is the almost exclusively occurring form in North Africa and in the Roma populations (Bönnemann C.G. et al., Neuromuscul. Disord. 1998;8:193-197; Dalichaouche I. et al., Muscle Nerve. 2017;56:129-135; Piccolo F. et al., Hum. Mol. Genet. 1996;5:2019-2022; Ben Othmane K. et al., Am. J. Hum. Genet. 1995;57:732-734).

LGMD2C (LGMD R5) is due to mutations in the γ-sarcoglycan (SGCG) gene coding for γ-sarcoglycan. SGCG is a single-pass transmembrane glycoprotein with a molecular weight of 35 kDa; it is composed of a small intracellular domain localized on the N terminus, a transmembrane domain and a large extracellular domain, containing N-glycosylation sites. Together with α-, β-, and δ-sarcoglycans, it forms part of the sarcoglycan subcomplex present in the striated muscles. This subcomplex is an important member of the dystrophin-associated glycoprotein complex (DGC), a crucial player in maintaining the linkage between the subsarcolemmal cytoskeleton and the extracellular matrix. Mutations in any of the sarcoglycans perturb the DGC complex formation, leading to a variable level of secondary deficiency of the other sarcoglycans on the sarcolemma. This destabilization of the complex induces a loss of stability in the sarcolemma and a loss of protection of muscle fibers from contraction-induced damage (Petrof B.J. et al., Proc. Natl. Acad. Sci. USA. 1993;90:3710-3714 ; Cohn R.D. and Campbell K.P, Muscle Nerve. 2000;23:1456-1471).

This loss of protection leads to the genetic defect in LGMD2C inducing a necrotic degenerative-regenerative process, resulting in progressive muscle wasting. The disease is characterized by predominant proximal muscle weakness in the limbs, almost always starting in the lower limbs, common calf hypertrophy, and early joint contractures. The frequency of respiratory insufficiency and dilated cardiomyopathy is variable. Clinical severity is usually correlated with the quantity of residual protein, and genotype-phenotype correlations can be observed. Null mutations are usually associated with absent proteins and severe Duchenne muscular dystrophy (DMD)-like phenotype, while missense mutations are associated with reduced amounts of protein and a milder LGMD-like phenotype (Semplicini C. et al., Neurology. 2015;84:1772-1781; Magri F. et al., Muscle Nerve. 2017;55:55-68).

To date, no treatment is available for LMGD2C.

Recently, a gene-therapy approach for the correction of the pathology was demonstrated in a mouse model deficient in γ-SG (Cordier L. et al., Mol. Ther. 2000;1: 119-129). In 2012, the result of a phase I-II clinical trial for LGMD2C of intramuscular injection of an AAV1 expressing the human γ-SG gene under the control of the desmin promoter was reported (Herson S. et al., Brain. 2012;135:483-492). Following this trial, Israeli et al. (Mol Ther Methods Clin Dev. 2019; 13:494-502) have reported the result of a dose-effect study focused on muscle restoration after systemic administration of an AAV2/8 harboring the same construct, i.e. expressing γ-SG under the control of the desmin promoter in Sgcg^{-/-} mice.

On another hand, document WO2019/152474 has disclosed a codon-optimized sequence encoding SGCG, harbored by an AAVrh74 vector and expressed under the control of the MHCK7 promoter.

Therefore, gene replacement therapy based on SGCG appears as a promising treatment of pathologies resulting from a SGCG deficiency. However, there is still a need of safe and efficient treatments.

In relation to gene therapy, a safe expression system is defined as one which ensures the production of a therapeutically effective amount of the protein in the target tissues, i.e. in the tissues wherein said protein is needed to cure the abnormalities linked to the deficiency of the native protein, without displaying any toxicity, especially in the essential and vital organs or tissues.

### BRIEF SUMMARY OF THE INVENTION

The present invention aims at alleviating or curing the devastating pathologies linked to a γ-sarcoglycan (SGCG) deficiency such as Limb-Girdle Muscular Dystrophy type 2C (LGMD2C), by providing an expression system which ensures the production of an adequate amount of the protein in the skeletal muscles and in the heart, i.e. a therapeutically effective amount which is not toxic.

Even if it is well established that a certain level of SGCG expression is required in the heart considering the observation of a cardiac phenotype in a relatively important number of patients (Calvo et al., Neuromuscul. Disord. 2000; 10(8):560-6; Van der Kooi et al., Heart 1998; 79(1):73-7), it is highly desirable to have an expression system which allows SGCG expression at an adequate level in the skeletal muscles without leading to an excessive overproduction in the heart so as to respect the endogenous balance and avoid any toxicity.

### Definitions

Unless otherwise defined, all technical and scientific terms used therein have the same meaning as commonly understood by one of ordinary skill in the art. The terminology used in the description is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” or “approximately” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “nucleotide sequence encoding an amino acid sequence” includes all nucleotide sequences that are degenerate versions of each other and that encode the same amino acid sequence. The phrase nucleotide sequence that encodes a protein or a RNA or a cDNA may also include introns to the extent that the nucleotide sequence encoding the protein may in some version contain an intron(s).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein’s or peptide’s sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A protein may be “altered” and contain deletions, insertions, or substitutions of amino acid residues which produce a silent change and result in a functionally equivalent. Deliberate amino acid substitutions may be made on the basis of similarity in polarity, charge, solubility, hydrophobicity, hydrophilicity, and/or the amphipathic nature of the residues as long as the biological activity is retained. For example, negatively charged amino acids may include aspartic acid and glutamic acid; positively amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

A “variant”, as used herein, refers to an amino acid sequence that is altered by one or more amino acids. The variant may have “conservative” changes, wherein a substituted amino acid has similar structural or chemical properties, e.g. replacement of leucine with isoleucine. A variant may also have “non-conservative” changes, e.g. replacement of a glycine with a tryptophan. Analogous minor variations may also include amino acid deletions or insertions, or both. Guidance in determining which amino acid residues may be substituted, inserted, or deleted without abolishing biological or immunological activity may be found using computer programs well known in the art.

“Identical” or “homologous” refers to the sequence identity or sequence similarity between two polypeptides or between two nucleic acid molecules. When a position in both of the two compared sequences is occupied by the same base or amino acid monomer subunit, e.g., if a position in each of two DNA molecules is occupied by adenine, then the molecules are homologous or identical at that position. The percent of homology/identity between two sequences is a function of the number of matching positions shared by the two sequences divided by the number of positions compared X 100. For example, if 6 of 10 of the positions in two sequences are matched then the two sequences are 60% identical. Generally, a comparison is made when two sequences are aligned to give maximum homology/identity.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one, which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell preferentially if the cell is a cell of the tissue type corresponding to the promoter.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics, which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. A subject can be a mammal, e.g. a human, a dog, but also a mouse, a rat or a nonhuman primate. In certain non-limiting embodiments, the patient, subject or individual is a human.

A “disease” or a “pathology” is a state of health of a subject wherein the subject cannot maintain homeostasis, and wherein if the disease is not ameliorated then the subject’s health continues to deteriorate. In contrast, a “disorder” in a subject is a state of health in which the subject is able to maintain homeostasis, but in which the subject’s state of health is less favorable than it would be in the absence of the disorder. Left untreated, a disorder does not necessarily cause a further decrease in the subject’s state of health.

A disease or disorder is “alleviated” or “ameliorated” if the severity of a symptom of the disease or disorder, the frequency with which such a symptom is experienced by a patient, or both, is reduced. This also includes halting progression of the disease or disorder. A disease or disorder is “cured” if the severity of a symptom of the disease or disorder, the frequency with which such a symptom is experienced by a patient, or both, is eliminated.

A “therapeutic” treatment is a treatment administered to a subject who exhibits signs of pathology, for the purpose of diminishing or eliminating those signs. A “prophylactic” treatment is a treatment administered to a subject who does not exhibit signs of pathology or has not be diagnosed for the pathology yet, for the purpose of preventing or postponing the occurrence of those signs.

As used herein, “treating a disease or disorder” means reducing the frequency or severity of at least one sign or symptom of a disease or disorder experienced by a subject. Disease and disorder are used interchangeably herein in the context of treatment.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

### DETAILED DESCRIPTION OF THE INVENTION

This invention is based on the identification by the inventors that the endogenous quantity of SGCG in the heart is generally similar or even inferior to that in the skeletal muscles. Therefore, an expression of SGCG produced from an expression system, which is much higher in the heart than in the skeletal muscles, could be deleterious and should be avoided.

This invention provides technical solutions for this newly identified problem, particularly regarding excessive cardiac expression besides the skeletal muscle expression of the SGCG transgene and more generally of sarcoglycans.

Thus and in general, this invention relates to an expression system for systemic administration comprising a sequence encoding gamma-sarcoglycan (SGCG) placed under the control of a promoter allowing an adequate expression of SGCG in the skeletal muscles and in the heart.

In other words, the invention concerns an expression system comprising a sequence encoding a SGCG protein, the said expression system allowing:
the expression at a therapeutically acceptable level of the protein in the target tissue(s), advantageously in the skeletal muscles and in the heart; but
the expression at an adequate level of the protein in the heart compared to its expression level in the skeletal muscles so as to avoid any potential cardiac toxicity.

In the frame of the invention, an expression system is generally defined as a polynucleotide which allows the in vivo production of SGCG. According to one aspect, said system comprises a nucleic acid encoding a SGCG protein, as well as the regulatory elements required for its expression, at least a promoter. Said expression system can then corresponds to an expression cassette. Alternatively, said expression cassette can be harboured by a vector or a plasmid. The wording “expression system” as used therein covers all aspects.

According to the invention, a target tissue is defined as a tissue or organ in which the protein is to play a therapeutic role, especially in cases where the native gene encoding this protein is defective. According to a particular embodiment of the invention, the target tissue includes the striated skeletal muscles, hereafter referred to as skeletal muscles i.e. all the muscles involved in motor ability and the diaphragm, and smooth muscles. Non limiting examples of target skeletal muscles are tibialis anterior (TA), gastrocnemius, soleus, quadriceps, psoas, deltoid, diaphragm, gluteus, extensorum digitorum longus (EDL), biceps brachii muscles, ...

As mentioned above, the heart can also be affected in various diseases linked to SGCG deficiencies and is therefore also a potential target tissue. However and in the frame of the present application, it is shown that SGCG when produced in too high quantity from existing expression systems can reach excessive levels, which may be toxic in the heart. Therefore and in relation to gene transfer, the expression system should be in favour of an adequate SGCG expression in the heart and in the skeletal muscles, preferentially comparable to the profile observed endogenously, i.e. with the native gene.

Then, even if SGCG has a therapeutic role to play in the heart, its level of expression should be tightly regulated since an excess of this protein in this tissue may be harmful or even fatal, and therefore toxic.

Thus and according to a particular aspect, the present invention relates to an expression system for systemic administration comprising a sequence encoding gamma-sarcoglycan (SGCG) placed under the control of a promoter allowing an adequate expression of SGCG in the skeletal muscles and in the heart.

According to a first characteristic, the expression system of the invention comprises a sequence encoding gamma-sarcoglycan (SGCG or γ-SG), corresponding to a transgene. In the context of the invention, the term “transgene” refers to a sequence, preferably an open reading frame, provided in trans using the expression system of the invention.

According to a particular embodiment, this sequence is a copy, identical or equivalent, of an endogenous sequence present in the genome of the body into which the expression system is introduced.

According to another embodiment, the endogenous sequence has one or more mutations rendering the protein partially or fully non-functional or even absent (lack of expression or activity of the endogenous protein), or not properly located in the desired subcellular compartment. In other words, the expression system of the invention is intended to be administered to a subject having a defective copy of the sequence encoding the protein and having an associated pathology.

Thus, the sequence carried by the expression system of the invention can be defined as encoding a protein having a therapeutic activity in the context of a pathology linked to a SGCG deficiency. The concept of therapeutic activity is defined as below in connection with the term “therapeutically acceptable level”.

The sequence encoding SGCG, also named ORF for “open reading frame”, is a nucleic acid sequence or a polynucleotide and may in particular be a single- or double-stranded DNA (deoxyribonucleic acid), an RNA (ribonucleic acid) or a cDNA (complementary deoxyribonucleic acid).

Advantageously, said sequence encodes a functional protein, i.e. a protein capable of ensuring its native or essential functions, especially in the skeletal muscles. This implies that the protein produced using the expression system of the invention is properly expressed and located, and is active.

According to a preferred embodiment, said sequence encodes the native protein, said protein being preferably of human origin. It may also be a derivative or a fragment of this protein, provided that the derivative or fragment retains the desired activity. Preferably, the term “derivative” or “fragment” refers to a protein sequence having at least 50%, preferably 60%, even more preferably 70% or even 80%, 85%, 90%, 95% or 99% identity with the human SGCG sequence. Proteins from another origin (non-human mammals, etc.) or truncated, or even mutated, but active proteins are for instance encompassed. Thus and in the context of the invention, the term “protein” is understood as the full-length protein regardless of its origin, as well as functional derivatives and fragments thereof.

The protein of interest in the context of the present invention is advantageously SGCG of human origin, even if e.g. the murine, rat or canine versions (which sequences are available in the databases) can be used.

According to a specific embodiment, a SGCG protein is a protein consisting of or comprising the amino acid sequence shown in SEQ ID NO: 1 (corresponding to a protein of 291 aa) or in SEQ ID NO: 2 which diverges from SEQ ID NO: 1 at one position (one residue) and corresponds to a natural variant thereof.

According to specific embodiments, SGCG is a protein having the same functions as the native human SGCG encoded by SEQ ID NO: 1 or SEQ ID NO: 2, especially the ability to interact with α-, β- and 8-sarcoglycans to form part of the sarcoglycan subcomplex, a member of the dystrophin-associated glycoprotein complex (DCG) and/or to alleviate, at least partially, one or more of the symptoms associated with a defect in SGCG, especially the LGMD2C phenotype as disclosed above. It can be a fragment and/or a derivative thereof. According to one embodiment, said SGCG sequence has identity greater than or equal to 50%, 60%, 70%, 80%, 90%, 95% or even 99% with sequence SEQ ID NO: 1 or SEQ ID NO: 2. As an example, Gao et al. (The Journal of Clinical Investigation, 2015; 125(11): 4186-95) have disclosed a so-called Mini-Gamma encoded by a mRNA wherein exons 4 to 7 have been skipped.

Any sequence encoding these proteins, functional therapeutic derivatives or fragments thereof, can be implemented as part of the expression system of the invention. By way of example, the corresponding nucleotide sequences (cDNA) are the sequences identified in WO2019/152474.

According to a specific embodiment, the sequence encoding SGCG comprises or consists of sequence SEQ ID NO: 3, or corresponds to nucleotides 1186 to 2061 of sequence SEQ ID NO: 5 or to nucleotides 1357 to 2232 of sequence SEQ ID NO: 6. Also of interest is any sequence having identity greater than or equal to 80%, 90%, 95% or even 99% with sequence SEQ ID NO: 3 and encoding a SGCG protein, preferably of sequence SEQ ID NO: 1 or SEQ ID NO: 2.

The present invention refers to a SGCG protein whose mutation causes a disease in one or more target tissues, especially in the skeletal muscles and possibly in the heart.

Mutations in the SGCG gene, in a known manner, can generate the entire range of pathologies named Limb-Girdle Muscular Dystrophy type 2C (LGMD2C or LGMD R5). Clinical severity is usually correlated with the quantity of residual protein, and genotype-phenotype correlations can be observed: Null mutations are usually associated with severe Duchenne muscular dystrophy (DMD)-like phenotype, while missense mutations are associated with a milder LGMD-like phenotype. Thus and according to the strategy for replacement or transfer of the gene, the provision in trans of a sequence encoding a therapeutic SGCG, which is for example native, helps to treat said pathologies.

According to the invention and advantageously, the expression system or the promoter present in said expression system must allow the expression at a therapeutically acceptable level of the SGCG protein in the skeletal muscles and possibly in the heart. According to a preferred embodiment and as reported in the present application, a therapeutically acceptable level of SGCG corresponds to at least 30% (0.3 times) of the quantity of the endogenous protein in the target tissues, especially in the skeletal muscles and possibly in the heart. In other words and advantageously, the ratio between the quantity of SGCG, especially in the skeletal muscles, and the quantity of endogenous SGCG in said tissue is superior or equal to 0.3, 0.4, 0.5, 0.6, 0.7, 0.8, 0.9 or 1, or can even reach 2, 3, 4, 5, 6, 7, 8, 9 or 10.

Moreover and according to another preferred embodiment, the expression system of the invention or the promoter present in said expression system must allow the expression of SGCG at a toxically acceptable level in the heart. According to a preferred embodiment and as reported in the present application, a toxically acceptable level of SGCG does not exceed 800% (8 times) of the quantity of the endogenous protein in the heart. In other words and advantageously, the ratio between the quantity of SGCG in the heart and the quantity of endogenous SGCG in said tissue is inferior or equal to 20, 15, 10 or 9, advantageously inferior or equal to 8, 7, 6, 5, 4, 3, 2 or even 1.

In the context of the invention, the term “protein expression” may be understood as “protein production”. Thus, the expression system must allow for both transcription and translation of the protein at the levels defined above. Also important is the correct folding and localisation of said protein.

The levels defined in the context of the invention, namely “therapeutically acceptable” and “toxically acceptable” are related to the amount or quantity of protein, as well as its activity as defined below.

The evaluation of the amount of protein produced in a given tissue can be carried out by immunodetection using an antibody directed against said protein, for example by Western blot or ELISA, or by mass spectrometry. Alternatively, the corresponding messenger RNAs may be quantified, for example by PCR or RT-PCR. This quantification can be performed on one sample of the tissue or on several samples. Thus and in the case where the target tissues are skeletal muscles, it may be carried out on a muscular type or several types of muscles (for example quadriceps, diaphragm, tibialis anterior, triceps, etc.).

In the context of the invention, the term “therapeutically acceptable level” refers to the fact that the protein produced from the expression system of the invention helps improve the pathological condition of the patient, particularly in terms of quality of life or lifespan. Thus and in connection with a disease affecting skeletal muscles, this involves improving the muscular condition of the subject affected by the disease or restoring a muscular phenotype similar to that of a healthy subject. As mentioned above, the muscular state, mainly defined by the strength, size, histology and function of the muscles, can be evaluated by different methods known in the art, e.g. biopsy, measurement of the strength, muscle tone, volume, or mobility of muscles, clinical examination, medical imaging, biomarkers, etc.

Thus, the criteria that help assess a therapeutic benefit as regards skeletal muscles and that can be evaluated at different times after the treatment are in particular at least one among:
increased life expectancy;
increased muscle strength
improved histology; and/or
improved functionality of the diaphragm.

In the context of the invention, the term “toxically acceptable level” refers to the fact that the protein produced from the expression system of the invention does not cause significant alteration of the tissue, especially histologically, physiologically and/or functionally. In particular, the expression of the protein may not be lethal. The toxicity in a tissue can be evaluated histologically, physiologically and functionally.

In the particular case of the heart, any toxicity of a protein can be evaluated by a study of the morphology and the heart function, by clinical examination, electrophysiology, imaging, biomarkers, monitoring of the life expectancy or by histological analysis, including the detection of fibrosis and/or cellular infiltrates and/or inflammation, for example by staining with sirius red or hematoxyline (e.g. Hematoxyline-Eosin-Saffran (HES) or Hematoxyline-Phloxin-Saffron (HFS)).

Advantageously, the level of efficacy and/or toxicity of the expression system according to the invention is evaluated in vivo in the animal, possibly in an animal having a defective copy of the gene encoding the protein and thus affected by the associated pathology. Preferably, the expression system is administered systemically, for example by intravenous (i.v.) injection.

According to the invention and preferably, the expression system comprises at least one sequence that allows an adequate expression of SGCG in the skeletal muscles and in the heart.

According to another embodiment, an expression system according to the invention comprises a sequence encoding gamma-sarcoglycan (SGCG) placed under the control of a promoter allowing an adequate expression of SGCG in the skeletal muscles and in the heart.

Suitably, an expression system of the invention comprises a promoter sequence governing the transcription of the sequence encoding the protein, preferably placed at 5′ of the transgene and functionally linked thereto. Preferably, this ensures a therapeutically acceptable level of expression of the protein in the skeletal muscles and possibly in the heart, as well as a toxically acceptable level in the heart, as defined above.

In a characteristic manner according to the invention, such a promoter should further ensure an adequate expression of SGCG in the heart and in the skeletal muscles, e.g. in the TA muscle.

In the frame of the invention, the term “adequate” is an equivalent of “appropriate”, “adapted” or “balanced” and advantageously means that the expression profile is comparable to the profile observed endogenously, i.e. with the native gene. As reported in the examples, the quantity of the SGCG protein in the heart should advantageously not exceed the quantity of the SGCG protein in the skeletal muscles. As already mentioned, said quantity can be evaluated by any technique known in the art, e.g. by evaluating the intensity of the corresponding band in western blotting.

As observed in relation to the endogenous gene, the quantity of SGCG produced from the expression system according to the invention in the skeletal muscles is advantageously superior or equal to the quantity produced in the heart.

This can be evaluated by calculating the ratio between the SGCG amount in the heart and the SGCG amount in the skeletal muscles, e.g. in the TA muscle.

According to an embodiment, this ratio should not exceed 5. Advantageously, this ratio should be less than or equal to 4, 3, 2, or even 1. More advantageously, this ratio is inferior to 1.

Conversely, said ratio can be expressed as the ratio between the SGCG amount in the skeletal muscles, e.g. in the TA muscle, and the SGCG amount in the heart.

According to an embodiment, this ratio should not be less than 0.2. Advantageously, this ratio is greater than or equal to 0.3, 0.4, 0.5, 0.6, 0.7, 0.8, 0.9 or even 1, 2, 3, 4, 5, 6, 7, 8, 9 or 10. More advantageously, this ratio is at least equal to 0.9 or even 1.

This may include inducible or constitutive, natural or synthetic (artificial) promoters. Similarly, they can be of any origin, including human, of the same origin as the transgene or of another origin.

This include any promoter displaying the above-defined expression profile in the skeletal muscles and in the heart, e.g.:
derivatives of the muscle creatine kinase promoter, especially a truncated MCK promoter with double (dMCK) or triple (tMCK) tandem of MCK enhancer, or the CK6 promoter (Wang et al., 2008, Gene Therapy, Vol. 15, pages 1489-99) ;
the muscle hybrid (MH) promoter (Piekarowicz et al., 2017, European Society Of Gene & Cell Therapy conference, poster P096; HUMAN GENE THERAPY 28:A44 (2017), DOI: 10.1089/hum.2017.29055.abstracts);
promoters containing at least one sequence USE (UpStream Enhancer) as e.g. identified in the troponin I promoter sequence (Corin et al., 1995, Proc. Natl. Acad. Sci., Vol. 92, pages 6185-89), or a 100-bp deletion thereof (ΔUSE; Blain et al., 2010, Human Gene Therapy, Vol. 21, pages 127-34), possibly in 3 (x3) or 4 (x4) copies. Of particular interest are the DeltaUSEx3 (DUSEx3) promoter and the DeltaUSEx4 (DUSEx4) promoter;
the promoter of the gamma-sarcoglycan gene;
the skeletal alpha-actin (ACTA1) promoter or derived versions thereof.

According to specific embodiments, such a promoter is not the desmin promoter, e.g. of sequence SEQ ID NO: 13, nor the CK8 promoter, e.g. of sequence SEQ ID NO: 14. According to another embodiment, such a promoter is not the MHCK7 promoter, e.g. as disclosed in WO2019/152474.

Advantageously, the promoter to be used in the frame of the invention is the tMCK promoter. According to a preferred embodiment, the tMCK promoter has the sequence as shown in SEQ ID NO: 4.

Promoter sequences derived from said sequences or corresponding to a fragment thereof but having a similar promoter activity, particularly in terms of tissue specificity and possibly effectiveness, are also covered under the present invention. Preferably, the term “derivative” or “fragment” refers to a sequence having at least 60%, preferably 70%, even more preferably 80% or even 90%, 95% or 99% identity with said sequences, advantageously with SEQ ID NO: 4. Of particular interest are the promoter sequences allowing an adequate SGCG expression in the heart and in the skeletal muscles as defined above.

According to a specific embodiment, the present invention therefore relates to an expression system comprising a sequence encoding SGCG, preferably of sequence SEQ ID NO: 3, placed under the control of a promoter having the sequence SEQ ID NO: 4, or a derivative or fragment thereof as defined above.

Advantageously, the expression system of the invention comprises a sequence corresponding to:
nucleotides 1 to 2061 of SEQ ID NO: 5; or
nucleotides 172 to 2232 of SEQ ID NO: 6.

According to a specific embodiment, the promoter of interest is further selected for its ability to allow a low expression or no expression in non-target tissues, i.e. in the tissues in which SGCG has no therapeutic effect or in which SGCG is not naturally expressed. As mentioned above and advantageously, muscles (smooth and skeletal) and heart are excluded from said non-target tissues. On the contrary, the liver can be considered as a non-target tissue.

According to a specific embodiment, the promoter allowing an adequate expression of SGCG in the skeletal muscles and in the heart has no activity or a low activity in non-target tissues, e.g. in the liver. Alternatively, the expression system according to the invention further comprises a sequence which allows preventing or decreasing SGCG expression in non-target tissues, especially in the liver.

In the context of the invention, the terminology “prevent the expression” preferably refers to cases where, even in the absence of the said sequence, there is no expression, while the terminology “decrease the level of expression” refers to cases where the expression is decreased (or reduced) by the provision of said sequence.

Advantageously, said sequence is capable of preventing the expression or reducing the level of expression of SGCG in the non-target tissues, wherein protein expression may be toxic or is not desired. This action may take place according to various mechanisms, particularly:
with regard to the level of transcription of the sequence encoding the protein;
with regard to transcripts resulting from the transcription of the sequence encoding the protein, e.g., via their degradation;
with regard to the translation of the transcripts into protein.

Such a sequence is preferably a target for a small RNA molecule e.g. selected from the following group:
microRNAs;
endogenous small interfering RNA or siRNAs;
small fragments of the transfer RNA (tRNA);
RNA of the intergenic regions;
Ribosomal RNA (rRNA);
Small nuclear RNA (snRNA);
Small nucleolar RNAs (snoRNA);
RNA interacting with piwi proteins (piRNA).

According to one embodiment, this sequence does not impact the SGCG expression in the target tissue(s), especially in the skeletal muscles and in the heart.

Preferably, such a sequence is selected for its effectiveness in the tissue wherein the expression of the protein has no therapeutic activity or is even toxic. Since the effectiveness of this sequence can be variable depending on the tissues, it may be necessary to combine several of these sequences, chosen for their effectiveness in said tissues.

According to a preferred embodiment, this sequence is a target sequence for a microRNA (miRNA). As known, such a judiciously chosen sequence helps to specifically suppress gene expression in selected tissues.

Thus and according to a particular embodiment, the expression system of the invention comprises a target sequence for a microRNA (miRNA) expressed or present in the tissue(s) in which the expression of the protein has no therapeutic activity and/or is toxic, e.g. in the liver. Suitably, the quantity of this miRNA present in the target tissue, especially the skeletal muscles and the heart, is less than that present in the tissues wherein SGCG is useless or even toxic, or this miRNA may not even be expressed in the target tissues. According to a particular embodiment, the target miRNA is not expressed in the skeletal muscles and possibly in the heart. According to another particular embodiment, it is specifically or even exclusively expressed in the liver.

As is known to the person skilled in the art, the presence or level of expression, particularly in a given tissue, of a miRNA may be assessed by PCR, preferably by RT-PCR, or by Northern blot.

Different miRNAs, as well as their target sequence and their tissue specificity, are known to those skilled in the art and are for example described in the document WO 2007/000668. MiRNAs expressed in the liver are e.g. miR-122.

According to a specific embodiment, the expression system according to the invention does not comprise any target sequence for a miRNA expressed in the heart, e.g. for miR208a.

According to the invention, an expression system or expression cassette comprises the elements necessary for the expression of the transgene present. In addition to sequences such as those defined above to ensure and to modulate transgene expression, such a system may include other sequences such as:
sequences for transcript stabilization, e.g. intron 2/exon 3 (modified) of the gene coding the human β globin (HBB2), e.g. corresponding to nucleotides 734 to 1179 of SEQ ID NO: 5 or 905 to 1350 of SEQ ID NO: 6. As shown in said sequences, said HBB2 intron is advantageously followed by consensus Kozak sequence (GCCACC) included before AUG start codon within mRNA, to improve initiation of translation;
a polyadenylation signal, e.g. the polyA of the gene of interest, the polyA of SV40 or of beta hemoglobin (HBB2), advantageously in 3′ of the sequence encoding SGCG. As a preferred example, the poly A of HBB2 corresponds to nucleotides 2072 to 2833 of SEQ ID NO: 5 or 2243 to 3004 of SEQ ID NO: 6;
enhancer sequences.

An expression system according to the invention can be introduced in a cell, a tissue or a body, particularly in humans. In a manner known to those skilled in the art, the introduction can be done ex vivo or in vivo, for example by transfection or transduction. According to another aspect, the present invention therefore encompasses a cell or a tissue, preferably of human origin, comprising an expression system of the invention.

The expression system according to the invention, in this case an isolated nucleic acid, can be administered in a subject, namely in the form of a naked DNA. To facilitate the introduction of this nucleic acid in the cells, it can be combined with different chemical means such as colloidal disperse systems (macromolecular complex, nanocapsules, microspheres, beads) or lipid-based systems (oil-in-water emulsions, micelles, liposomes).

Alternatively and according to another preferred embodiment, the expression system of the invention comprises a plasmid or a vector. Advantageously, such a vector is a viral vector. Viral vectors commonly used in gene therapy in mammals, including humans, are known to those skilled in the art. Such viral vectors are preferably chosen from the following list: vector derived from the herpes virus, baculovirus vector, lentiviral vector, retroviral vector, adenoviral vector and adeno-associated viral vector (AAV).

According to a specific embodiment of the invention, the viral vector containing the expression system is an adeno-associated viral (AAV) vector.

Adeno-associated viral (AAV) vectors have become powerful gene delivery tools for the treatment of various disorders. AAV vectors possess a number of features that render them ideally suited for gene therapy, including a lack of pathogenicity, moderate immunogenicity, and the ability to transduce post-mitotic cells and tissues in a stable and efficient manner. Expression of a particular gene contained within an AAV vector can be specifically targeted to one or more types of cells by choosing the appropriate combination of AAV serotype, promoter, and delivery method.

In one embodiment, the encoding sequence is contained within an AAV vector. More than 100 naturally occurring serotypes of AAV are known. Many natural variants in the AAV capsid exist, allowing identification and use of an AAV with properties specifically suited for dystrophic pathologies. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

As mentioned above, the use of AAV vectors is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector. Other currently used AAV serotypes include AAV1, AAV3, AAV4, AAV5, AAV6, AAV7, AAV8, AAV9, AAVrh10, AAVrh74, AAV 11 and AAV12. In addition, non-natural engineered variants and chimeric AAV can also be useful.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells.

Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, include AAV2/8 (US 7,282,199), AAV⅖ (available from the National Institutes of Health), AAV2/9 (WO2005/033321), AAV2/6 (US 6,156,303), AAVrh10 (WO2003/042397), AAVrh74 (WO2003/123503), AAV9-rh74 hybrid or AAV9-rh74-P1 hybrid (WO2019/193119), AAV variants disclosed in PCT/EP2020/061380 among others. In one embodiment, the vectors useful in the compositions and methods described herein contain, at a minimum, sequences encoding a selected AAV serotype capsid, e.g., an AAV8 capsid, or a fragment thereof. In another embodiment, useful vectors contain, at a minimum, sequences encoding a selected AAV serotype rep protein, e.g., AAV8 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV8 origin. Alternatively, vectors may be used in which the rep sequences are from an AAV serotype, which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 (US 7,282,199).

According to one embodiment, the composition comprises an AAV of serotype 2, 5, 8 or 9, or an AAVrh74. Advantageously, the claimed vector is an AAV8 or AAV9 vector, especially an AAV2/8 or AAV2/9 vector.

In the AAV vectors used in the present invention, the AAV genome may be either a single stranded (ss) nucleic acid or a double stranded (ds) / self complementary (sc) nucleic acid molecule.

Advantageously, the polynucleotide encoding SGCG is inserted between the ITR (« Inverted Terminal Repeat ») sequences of the AAV vector. Typical ITR sequences correspond to nucleotides 1 to 145 of SEQ ID NO: 6 (5′ITR sequences) and to nucleotides 3005 to 3149 of SEQ ID NO: 6 (3′ITR sequences).

Recombinant viral particles can be obtained by any method known to the one skilled in the art, e.g. by co-transfection of 293 HEK cells, by the herpes simplex virus system and by the baculovirus system. The vector titers are usually expressed as viral genomes per mL (vg/mL).

In one embodiment, the vector comprises regulatory sequences, especially a promoter sequence, advantageously as described above.

In relation to a polynucleotide encoding the sequence SEQ ID NO: 1, a vector of the invention may comprise the sequence shown in SEQ ID NO: 5 or SEQ ID NO: 6.

According to a preferred embodiment, the expression system of the invention includes a vector having a suitable tropism, in this case higher for the target tissue(s), advantageously the skeletal muscles and the heart, and possibly the smooth muscles, than for the tissues where the expression of the protein could be toxic.

Further aspects of the invention concern:
A cell comprising the expression system of the invention or a vector comprising said expression system, as disclosed above.
The cell can be any type of cells, i.e. prokaryotic or eukaryotic. The cell can be used for propagation of the vector or can be further introduced (e.g. grafted) in a host or a subject. The expression system or vector can be introduced in the cell by any means known in the art, e.g. by transformation, electroporation or transfection. Vesicles derived from cells can also be used.
A transgenic animal, advantageously non-human, comprising the expression system of the invention, a vector comprising said expression system, or a cells comprising said expression system or said vector, as disclosed above.

Another aspect of the invention relates to a composition comprising an expression system, a vector or a cell, as disclosed above, for use as a medicament.

According to an embodiment, the composition comprises at least said gene therapy product (the expression system, the vector or the cell), and possibly other active molecules (other gene therapy products, chemical molecules, peptides, proteins...), dedicated to the treatment of the same disease or another disease.

According to a specific embodiment, the use of the expression system according to the invention is combined with the use of anti-inflammatory drugs such as corticoids.

The present invention then provides pharmaceutical compositions comprising an expression system, a vector or a cell of the invention. Such compositions comprise a therapeutically effective amount of the therapeutic (the expression system or vector or cell of the invention), and a pharmaceutically acceptable carrier. In a specific embodiment, the term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, sustained-release formulations and the like. Examples of suitable pharmaceutical carriers are described in “Remington’s Pharmaceutical Sciences” by E. W. Martin. Such compositions will contain a therapeutically effective amount of the therapeutic, preferably in purified form, together with a suitable amount of carrier so as to provide the form for proper administration to the subject.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to release pain at the site of the injection.

In one embodiment, the composition according to the invention is suitable for administration in humans. The composition is preferably in a liquid form, advantageously a saline composition, more advantageously a phosphate buffered saline (PBS) composition or a Ringer-Lactate solution.

The amount of the therapeutic (i.e. an expression system or a vector or a cell) of the invention which will be effective in the treatment of the target diseases can be determined by standard clinical techniques. In addition, in vivo and/or in vitro assays may optionally be employed to help predict optimal dosage ranges. The precise dose to be employed in the formulation will also depend on the route of administration, the weight and the seriousness of the disease, and should be decided according to the judgment of the practitioner and each patient’s circumstances.

Suitable administration should allow the delivery of a therapeutically effective amount of the gene therapy product to the target tissues, especially skeletal muscles and possibly heart. In the context of the invention, when the gene therapy product is a viral vector comprising a polynucleotide encoding human SGCG, the therapeutic dose is defined as the quantity of viral particles (vg for viral genomes) containing the SGCG sequence, administered per kilogram (kg) of the subject.

Available routes of administration are topical (local), enteral (system-wide effect, but delivered through the gastrointestinal (GI) tract), or parenteral (systemic action, but delivered by routes other than the GI tract). The preferred route of administration of the compositions disclosed herein is parenteral which includes intramuscular administration (i.e. into the muscle) and systemic administration (i.e. into the circulating system). In this context, the term “injection” (or “perfusion” or “infusion”) encompasses intravascular, in particular intravenous (IV), intramuscular (IM), intraocular, intrathecal or intracerebral administration. Injections are usually performed using syringes or catheters.

In one embodiment, systemic delivery of the composition comprises administering the composition near a local treatment site, i.e. in a vein or artery nearby a weakened muscle. In certain embodiments, the invention comprises the local delivery of the composition, which produces systemic effects. This route of administration, usually called “regional (loco-regional) infusion”, “administration by isolated limb perfusion” or “high-pressure transvenous limb perfusion” has been successfully used as a gene delivery method in muscular dystrophy.

According to one aspect, the composition is administered to an isolated limb (loco-regional) by infusion or perfusion. In other words, the invention comprises the regional delivery of the composition in a leg and/or arm by an intravascular route of administration, i.e. a vein (transvenous) or an artery, under pressure. This is usually achieved by using a tourniquet to temporarily arrest blood circulation while allowing a regional diffusion of the infused product, as e.g. disclosed by Toromanoff et al. (2008).

In one embodiment, the composition is injected in a limb of the subject. When the subject is a human, the limb can be the arm or the leg. According to one embodiment, the composition is administered in the lower part of the body of the subject, e.g. below the knee, or in the upper part of the body of the subject, e.g., below the elbow.

A preferred method of administration according to the invention is systemic administration. Systemic injection opens the way to an injection of the whole body, in order to reach the entire muscles of the body of the subject including the heart and the diaphragm and then a real treatment of these systemic and still incurable diseases. In certain embodiments, systemic delivery comprises delivery of the composition to the subject such that composition is accessible throughout the body of the subject.

According to a preferred embodiment, systemic administration occurs via injection of the composition in a blood vessel, i.e. intravascular (intravenous or intra-arterial) administration. According to one embodiment, the composition is administered by intravenous injection, through a peripheral vein.

The systemic administration is typically performed in the following conditions:
a flow rate of between 1 to 10 mL/min, advantageously between 1 to 5 mL/min, e.g. 3 mL/min;
the total injected volume can vary between 1 and 20 mL, preferably 5 mL of vector preparation per kg of the subject. The injected volume should not represent more than 10% of total blood volume, preferably around 6%.

When systemically delivered, the composition is preferably administered with a dose less than or equal to 10¹⁵ vg/kg or even 10¹⁴ vg/kg, advantageously superior or equal to 10¹⁰, 10¹¹, or even 10¹² vg/kg. Specifically, the dose can be between 5.10¹² vg/kg and 10¹⁴ vg/kg, e.g. 1, 2, 3, 4, 5, 6, 7, 8 or 9.10¹³ vg/kg. A lower dose of e.g. 1, 2, 3, 4, 5, 6, 7, 8 or 9.10¹² vg/kg can also be contemplated in order to avoid potential toxicity and /or immune reactions. As known by the skilled person, a dose as low as possible giving a satisfying result in term of efficiency is preferred.

In a specific embodiment, the treatment comprises a single administration of the composition.

Such compositions are notably intended for gene therapy, particularly for the treatment of Limb-Girdle Muscular Dystrophy type 2C (LGMD2C or LGMD R5) or γ-sarcoglycanopathy in a subject.

Subjects that could benefit from the compositions of the invention include all patients diagnosed with such a disease or at risk of developing such a disease. A subject to be treated can then be selected based on the identification of mutations or deletions in the SGCG gene by any method known to the one skilled in the art, including for example sequencing of the SGCG gene, and/or through the evaluation of the SGCG level of expression or activity by any method known to the one skilled in the art. Therefore, said subjects include both subjects already exhibiting symptoms of such a disease and subjects at risk of developing said disease. In one embodiment, said subjects include subjects already exhibiting symptoms of such a disease. In another embodiment, said subjects are ambulatory patients and early non-ambulant patients.

More generally and according to further embodiments, an expression system according to the invention is useful for:
increasing muscular force, muscular endurance and/or muscle mass in a subject;
reducing fibrosis in a subject;
reducing contraction-induced injury in a subject;
treating muscular dystrophy in a subject;
reducing degenerating fibers or necrotic fibers in a subject suffering from muscular dystrophy;
reducing inflammation in a subject suffering from muscular dystrophy;
reducing levels of creatine kinase (or any other dystrophic marker) in a subject suffering from muscular dystrophy;
treating myofiber atrophy and hypertrophy in a subject suffering from muscular dystrophy;
decreasing dystrophic calcification in a subject suffering from muscular dystrophy;
decreasing fatty infiltration in a subject;
decreasing central nucleation in a subject.

According to one embodiment, the present invention concerns a method for treating such conditions comprising administering to a subject the gene therapy product (expression system, vector or cell) as disclosed above.

Advantageously, the expression system is administered systemically in the body, particularly in an animal, advantageously in mammals and more preferably in humans.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The disclosures of each and every patent, patent application, and publication cited herein are hereby incorporated herein by reference in their entirety.

Without further description, it is believed that one of ordinary skill in the art can, using the preceding description and the following illustrative examples, make and utilize the compounds of the present invention and practice the claimed methods.

### EXPERIMENTAL EXAMPLES

The invention is further described in detail by reference to the following experimental examples and the attached figures. These examples are provided for purposes of illustration only, and are not intended to be limiting.

In particular, the invention is illustrated in relation to an AAV8 vector comprising a sequence encoding SGCG placed under the control of the tMCK promoter.

### FIGURES

FIG. 1: A/ Western blot detection of γ-sarcoglycan (SGCG) expression in the tibialis anterior (TA) muscle and the heart of a mouse or a macaca using a γ-sarcoglycan antibody (Ab203113-Abcam) B/ Graphical presentation of SGCG expression in each tissue based on the signals detected in (A). Statistical ANOVA test: (*) indicates a P value of less than 0.05 (statistically significant). ns: non significant

FIG. 2: Luciferase activity of GFP-Luc transgene normalized by total protein amount in TA muscle and heart from C57B16 albino mice injected with AAV9-prom-GFP-Luc (Des, CK8 and tMCK).

FIG. 3: Vector genome copy number (VGCN) per diploid genome measured by QPCR in tissues (TA, heart and liver) from 3 groups of Sgcg-/- mice intravenously injected with an AAV8 vector harboring SGCG under the control of the desmin promoter (AAV8-Des-SGCG) or the CK8 promoter (AAV8-CK8-SGCG) or the tMCK promoter (AAV8-tMCK-SGCG).

FIG. 4: A/SGCG mRNA normalized by P0 endogenous level measured by RT-QPCR in tissues (TA, heart and liver) from the 3 groups of Sgcg-/- mice intravenously injected with an AAV8 vector harboring SGCG under the control of the desmin promoter (AAV8-Des-SGCG) or the CK8 promoter (AAV8-CK8-SGCG) or the tMCK promoter (AAV8-tMCK-SGCG). B/ Ratio between the relative abundance of SGCG/P0 mRNA and the VGCN in each tissue. C/ Ratio of the relative abundance of SGCG mRNA in heart versus TA muscle. The dotted line corresponds to a ratio of 1 (same expression level in heart and TA muscle). Statistical ANOVA test: (*) indicates a P value of less than 0.05 (statistically significant).

FIG. 5: A/ Western blot detection of human γ-sarcoglycan expression in the TA muscle and the heart of the 5 mice of each group (Sgcg-/- mice intravenously injected with an AAV8 vector harboring SGCG under the control of the desmin promoter (AAV8-Des-SGCG) or the CK8 promoter (AAV8-CK8-SGCG) or the tMCK promoter (AAV8-tMCK-SGCG)), using a human-specific γ-sarcoglycan antibody (Ab203112-Abcam). B/ Graphical presentation of SGCG expression in each tissue (Ht: heart; TA: tibialis anterior) based on the signals detected in (A). Statistical ANOVA test: (*) indicates a P value of less than 0.05 (statistically significant). ns: non significant

FIG. 6: Immunostaining anti-SGCG performed in TA and heart of Sgcg-/- mice intravenously injected with an AAV8 vector harboring SGCG under the control of the desmin promoter (AAV8-Des-SGCG) or the CK8 promoter (AAV8-CK8-SGCG) or the tMCK promoter (AAV8-tMCK-SGCG). Scale bar = 100 µm.

FIG. 7: A/ Graphic correlation between the percentage of SGCG expression and the percentage of centronucleated fibers. The black dots correspond to muscle from WT mice and the white ones from KO-Sgcg mice. The grey dots correspond to muscle from KO-Sgcg injected with different level of AAV transduction efficiency (5e12 vg/kg, 1e13 vg/kg and 5e13 vg/kg of AAV8-Des-SGCG) B/ Western blot detection of γ-sarcoglycan expression in the TA muscle and the heart of WT mice intravenously injected with PBS or AAV8-Des-SGCG (3^{e}14 vg/kg) using a γ-sarcoglycan antibody (Ab203113-Abcam) C/ Graphical presentation of SGCG expression in each tissue (Ht: heart; TA: tibialis anterior) based on the signals detected in (B).

FIG. 8: A/ Western blot detection of human γ-sarcoglycan expression in the TA muscle and the heart of rats of each group (Sprague dawley intravenously injected with an AAV8 vector harboring SGCG under the control of the tMCK promoter (AAV8 tMCK), the desmin promoter (AAV8 Desmin) and the MHCK7 promoter (AAV8 MHCK7), using a human-specific γ-sarcoglycan antibody (Ab203112-Abcam). B/ Graphical presentation of SGCG expression in each tissue (Heart; TA: tibialis anterior) based on the signals detected in (A). Statistical Student test: (*) indicates a P value of less than 0.05, (***) indicates a P value of less than 0.001 (statistically significant) ns: non significant

FIG. 9: Molecular ratio rMyh6 / rMyh7 measured by RT-QPCR transcripts in heart from the 3 groups of Sprague Dawley rat intravenously injected with PBS or, with an AAV8 vector harboring SGCG under the control of the tMCK promoter (AAV8-tMCK-SGCG),the desmin promoter (AAV8-Desmin-SGCG) and the MHCK7 promoter (AAV8-MHCK7-SGCG). Statistical ANOVA test: (**) indicates a P value of less than 0.001.

### MATERIALS AND METHODS

### Animal Models

The animal studies were performed in accordance to the current European legislation on animal care and experimentation (2010/63/EU) and approved by the institutional ethics committee of the Centre d′Exploration et de Recherche Fonctionnelle Expérimentale in Evry, France (protocol APAFIS DAP 2018-024-B#19736).

The Sgcg^{-/-} mouse strain (Hack et al., J. Cell. Biol. 1998;142:1279-87) was used in this study. These mice were bred in a pure C57BL/6J background by crossing 10 times onto the C57BL/6J background. The C57B⅙J and C57B16 albino mice were ordered to the Charles River Facility. Samples from macaca were provided by Inserm UMR 1089, Atlantic Gene Therapies, Institut de Recherche Thérapeutique (IRT 1) Université de Nantes (France) and Silabe (67207 Niederhausbergen, France). One-month-old male Sprague Dawley rats were also used in this study.

### Expressing Cassette and AAV-mediated Gene Transfer

Three different AAV cassettes were designed using the same ITR sequences, transgene GFP-Luc and polyA HBB2. The promoter was the only element that differs between the constructs. In this study, the human desmin (Des) promoter (SEQ ID NO: 13), the CK8 promoter (Goncalves et al., Mol Ther. 2011;19(7): 1331-41; SEQ ID NO: 14) and the tMCK promoter (Wang et al., Gene Therapy 2008;15:1489-99; SEQ ID NO: 4) were compared. The serotype 9 was used for the production of GFP-Luc recombinant adeno-associated virus (AAV9-prom-GFP-Luc).

Three other AAV cassettes were also designed using the same promoters but with the SGCG transgene (see SEQ ID NO: 6 in relation to the tMCK promoter). Moreover, the MHCK7 promoter as disclosed in WO2019/152474 (SEQ ID NO: 15) was further tested in this context. The serotype 8 was used for the production of recombinant SGCG adeno-associated virus (AAV8-prom-SGCG).

Viral genomes were quantified by a TaqMan™ real-time PCR assay using the primer pairs and TaqMan™ probes specific for the polyA HBB2 sequence:

FWD: 5′-CCAGGCGAGGAGAAACCA-3′ (SEQ ID NO: 7),

REV: 5′-CTTGACTCCACTCAGTTCTCTTGCT-3′ (SEQ ID NO: 8
), and

Probe: 5′-CTCGCCGTAAAACATGGAAGGAACACTTC-3′ (SEQ ID
NO: 9).

The different vectors were injected by a single systemic administration in the tail vein in order to express the GFP-Luc transgene in male one month-old C57B16 Albino mice or to restore γ-sarcoglycan expression in muscle of female five week-old Sgcg-/- mice. The doses of vector injected were normalized by the body’s weight of mice at 5el3vg/kg of AAV9-prom-GFP-Luc or at 5e12 vg/kg, 1e13 vg/kg 5e13 vg/kg or 3e14 vg/kg of AAV8-prom-SGCG. Three or two weeks after treatment, respectively, mice were sacrificed and tissues collected. The tibialis anterior (TA) muscle was chosen as a representative skeletal muscle.

Besides, one month old male Sprague Dawley rats were injected intravenously into the tail vein with the three AAV8 vectors MHCK7-hSGCG, Desmin-hSGCG and tMCK-hSCGC at a dose of 3e14vg/kg. Another rat group injected with PBS was also included as a control. One month after the injection, the rats were sacrified. The heart and the tibialis anterior (TA) muscles were collected.

### Quantification of the Luciferase by Luciferase Assay

Samples were first homogenized with 500 µL of assay buffer (Tris/Phosphate, 25 mM; Glycerol 15%; DTT, 1 mM; EDTA 1 mM; MgC12 8 mM) with 0.2% of Triton X-100 and Protease inhibitor cocktail PIC (Roche). Ten µl of lysate were loaded into flat-bottomed wells of a white opaque 96-well plate. The Enspire spectrophotometer was used for quantification of the luminescence. The pumping system delivers D-luciferin (167 µM; Interchim) and assay buffer with ATP (40 nM) (Sigma-Aldrich) to each well of the plate. The signal of Relative Light Unit (RLU) was measured after each dispatching of D-luciferin and ATP, respecting 2 sec delay between each samples. A BCA protein quantification (Thermo Scientific) was performed to normalize the quantity of protein in each sample. The result was expressed as the level of RLU normalized by the protein amount.

### Histological and Immunohistochemistry Analyses

Eight micrometers transversal cryosections were cut from liquid nitrogen-cooled isopentane frozen TA muscles or hearts. The transverse cryosections were then blocked with PBS containing 20% Fetal calf serum (FCS) for 1 h and incubated overnight at 4° C. with a rabbit monoclonal primary antibody directed against the human γ-sarcoglycan protein (Abcam - ab203112). After washing with PBS, sections were incubated with a goat anti-rabbit secondary antibody conjugated with AlexaFluor 594 dyes (Thermo Fisher Scientific) for 1h at room temperature.

After washing with PBS, sections were mounted with Fluoromount-G and DAPI (SouthernBiotech), and visualized on a fluorescence microscope (Zeiss - Zeiss Axiophot 2). A complete image acquisition of all sections was finally carried out using the AXIOSCAN microscope (Zeiss).

For determining the number of centronucleated fibers, the sections were labelled with a rabbit anti-laminin antibody (DAKO-Z0097), using a goat anti-rabbit antibody conjugated with AlexaFluor 488 dyes (Thermo Fisher Scientific) as secondary antibody and mounted with Fluoromount-G and DAPI (SouthernBiotech). Image acquisition of all sections was finally carried out using the AXIOSCAN microscope (Zeiss). The morphometric analyses of the skeletal muscles to define the number of centronuclear fibres (CNF/mm²) were performed as followed:

Scanned RGB images containing 8bits channels of the laminin Immunofluorescence and DAPI staining captured at l0x magnification are processed using the FIJI software for nuclei and fibers segmentation. Nuclei segmentation is performed based on the DAPI intensity using global thresholding (IsoData) and particles analysis. Fibers are segmented based on the laminin staining using the MorphoLib plugin ‘morphological segmentation’ tool (border image option) and ImageJ particles analysis tool (object circularity > .2, object size filter depending on muscle type and species).

Nuclei and fibers Regions of Interest (ROI) are converted to spatial objects using the R software (RImageJROI, spatstat and sp libraries) and intra-fiber nuclei identified by intersection of nuclei and fibers objects. For intra-fiber nuclei, their distance to the fiber center of gravity and closest membrane point is calculated.

Size, shape, fluorescence intensity filtering are performed to exclude artefacts (nerves identified as fiber, spited or merged fibers ... ).

Centro nucleated fibers are identified based on the distance between the nucleus and the closest membrane (relative to fiber Feret diameter or absolute distance, user’s choice).

### Viral Genome Copy Numbers (VGCN) Measurement in Tissues

Genomic DNA was extracted from frozen tissues using the NucleoMag Pathogen kit (Macherey Nagel) with the KingFisher robot (Thermo Fisher Scientific) according to manufacturer instructions. Vector genome copy number was determined using qPCR from 20 ng of genomic DNA. A serial dilution of a DNA sample of a plasmid harboring one copy of each amplicon was used as standard curve. Real-time PCR was performed using LightCycler480 (Roche Roche) with 0.2 µM of each primer and 0.1 µM of the probe according to the protocol of Absolute QPCR Rox Mix (Thermo Fisher Scientific). A sequence located in the polyA HBB2 of the cassette was used for the quantification of viral genome. The primer pairs and Taqman™ probes specific for the polyA HBB2 sequence were the same as disclosed above (SEQ ID NO: 7 to 9).

The ubiquitous acidic ribosomal phosphoprotein (P0) was used for genomic DNA quantification. Primer pairs and Taqman™ probe used for P0 amplification were:

FWD: 5′-CTCCAAGCAGATGCAGCAGA-3′ (SEQ ID NO: 10),

REV: 5′-ATAGCCTTGCGCATCATGGT-3′ (SEQ ID NO: 11), a
nd

Probe: 5′-CCGTGGTGCTGATGGGCAAGAA-3′ (SEQ ID NO: 12
).

The number of diploid genomes is half of the number of copies of P0 gene. The level of transduction of the tissue is determined by the VGCN per diploid genome.

### mRNA Quantification

Total RNA extraction was performed from frozen tissues following NucleoSpin® RNA Set for NucleoZOL protocol (Macherey Nagel). Extracted RNA was eluted in 60µl of RNase-free water and treated with TURBO™ DNase kit (Ambion) to remove residual DNA. Total RNA was quantified using a Nanodrop spectrophotometer (ND8000 Labtech).

For quantification of the transgene expression, one µg of RNA was reverse-transcribed using the RevertAid H minus Reverse transcriptase kit (Thermo Fisher Scientific) and a mixture of random oligonucleotides and oligo-dT. Real-time PCR was performed using LightCycler480 (Roche) using commercial sets of primers and probes for the quantification of human γ-sarcoglycan (Hs00165089_ml; Thermo Fisher Scientific). For mouse samples, the ubiquitous acidic ribosomal phosphoprotein (P0) was used to normalize the data across samples as well as the VGCN quantification described previously.

Each experiment was performed in duplicate. Quantification cycle (Cq) values were calculated with the LightCycler® 480 SW 1.5.1 using 2nd Derivative Max method. RT-qPCR results, expressed as raw Cq, were normalized to P0. The relative expression was calculated using the 2^{-ΔCt} Livak method.

### Measurement of the Transcript Ratio Myh6/Myh7

The transcripts of Myh6 and Myh7 were quantified by RT-QPCR using commercial sets of primers and probes for the quantification of rMyh6 (Rn00691721_g1; Thermo Fisher Scientific) and rMyh7 (Rn01488777_g1; Thermo Fisher Scientific). The result is expressed as a molecular ratio of the transcripts Myh6 versus Myh7.

### Western Blot Analysis

Frozen sections of approximately 1 mm of tissues (Liver, Heart or TA muscle) were solubilized in radio immunoprecipitation assay (RIPA) buffer with protease inhibitor cocktail. Protein extract was quantified by BCA (bicinchoninic acid) protein assay (Pierce). Thirty µg of total protein were processed for western blot analysis, using an anti-γ-sarcoglycan antibody (human-specific: Ab203112 and for common recognition of mouse human and macaca form :Abcam; Ab203113).

Fluorescence signal of the secondary antibodies was read on an Odyssey imaging system, and band intensities were measured by the Odyssey application software (LI-COR Biosciences, 2.1 version).

### Statistical Analyses

Statistical analyses were performed using the GraphPad Prism version 6.04 (GraphPad Software, San Diego, CA). Statistical analyses were performed using the statistical ANOVA or Sudent test as indicated. Data were expressed as mean ± SD. P values of less than 0.05 were considered statistically significant (*).

### RESULTS

### I/ Endogenous SGCG Expression Profile in Mouse and Macaca

In order to define the relative proportion of the endogenous SGCG between heart and skeletal muscle in different species, the relative abundance of the SGCG protein was investigated in different tissues (TA muscle as a representative of the skeletal muscles and the heart) of wild type mice or macaca.

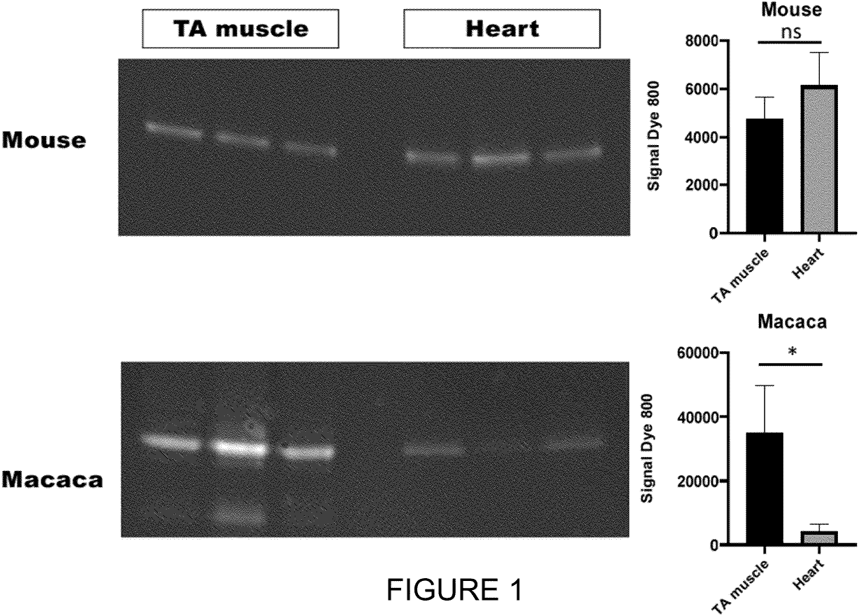

FIG. 1 reveals that in mice, SGCG is produced at a similar level in the TA muscle and in the heart. In the macaca, which is a mammalian model for humans, it is observed that the quantity of SGCG in the heart is drastically inferior to the SGCG quantity in the TA muscle.

### II/ Evaluation of Different Promoters in C57BL6 Mice

A study was performed to identify an expression construct displaying an expression profile in heart and TA muscle, similar as much as possible to that observed with the endogenous gene, i.e. with an expression at a similar level or even higher in the TA muscle than in the heart.

For this purpose, different promoters known to have a muscular activity have been tested using the reporter gene GFP-Luc.

Experiments were performed to compare the desmin promoter, the CK8 promoter and the tMCK promoter. The desmin promoter was chosen because it corresponds to the one tested by Israeli et al. (Mol Ther Methods Clin Dev. 2019; 13:494-502) who have reported its efficiency for restoring muscular activity.

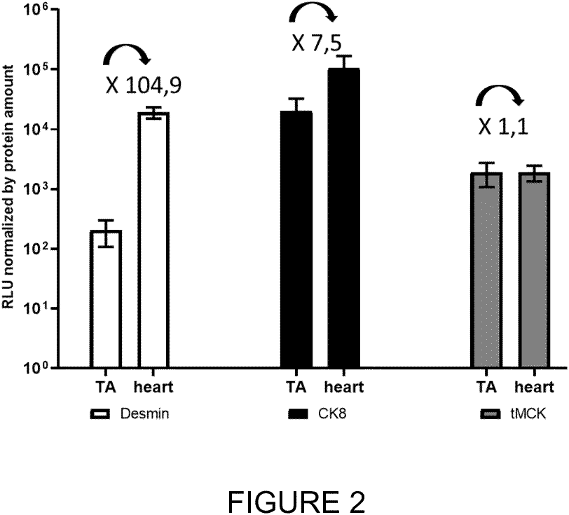

FIG. 2 reveals that:
The AAV9-CK8-GFP-Luc vector is the more efficient to transduce both heart and TA muscle;
The AAV9-tMCK-GFP-Luc appear to be weaker in terms of promoter strength but more equilibrated between heart and skeletal muscle expression.

It clearly appears that the tMCK promoter is a promising candidate, ensuring an adequate expression in in heart and TA muscle, as observed with the endogenous gene in the mouse and macaca. On the contrary, the desmin and CK8 promoters give rise to a very high expression in heart, superior to that observed in the TA muscle, with a possible associated cardiac toxicity.

### III/ Validation of the tMCK Promoter in Sgcg-/- Mice

To validate these observations, further studies were performed to compare 3 different SGCG AAV8 vectors intravenously injected in SGCG deficient mice. The promising tMCK promoter was compared to the two other promoters as tested above, i.e. the Desmin promoter and the CK8 promoter.

First, the efficacy of transduction was compared between the 3 constructs.

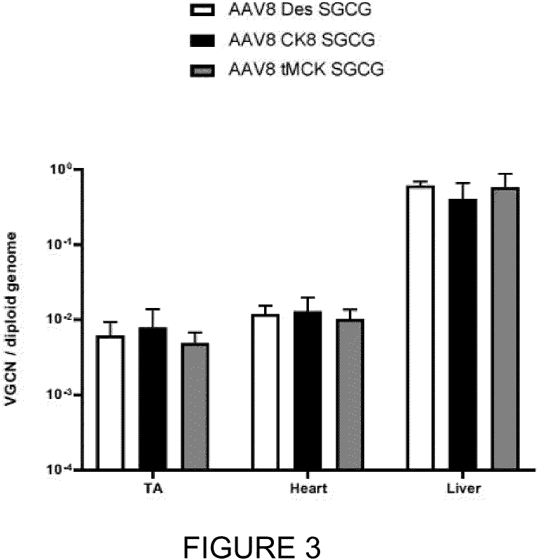

As shown by FIG. 3, there is no bias regarding the infectiosity of the 3 vectors since they transduced at the same level the same tissues. The liver was clearly the organ the most transduced (~1 VGCN / diploid genome). The similar transduction of heart and TA muscle reached around 0.01 VGCN / diploid genome. With this low level of infection, there was no risk to reach a saturation effect that could interfere with the following analyses.

Then, the transcriptional activity of the 3 promoters was compared.

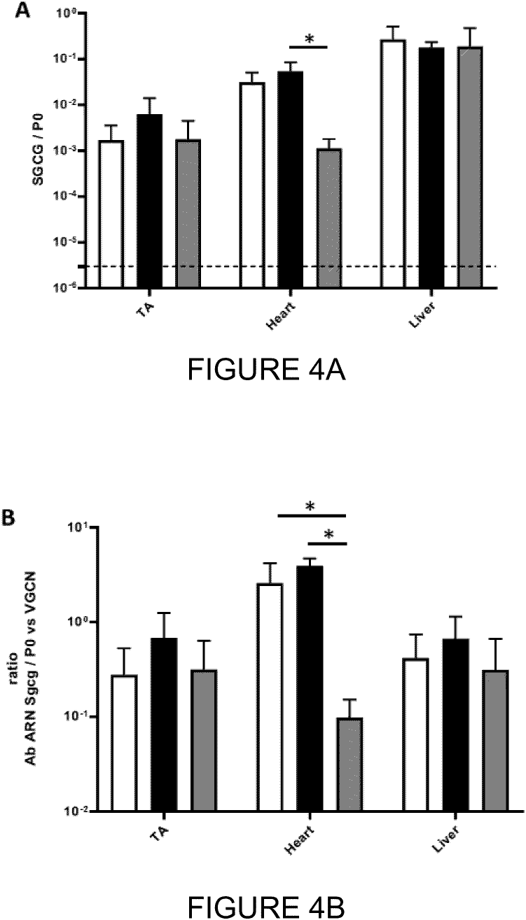

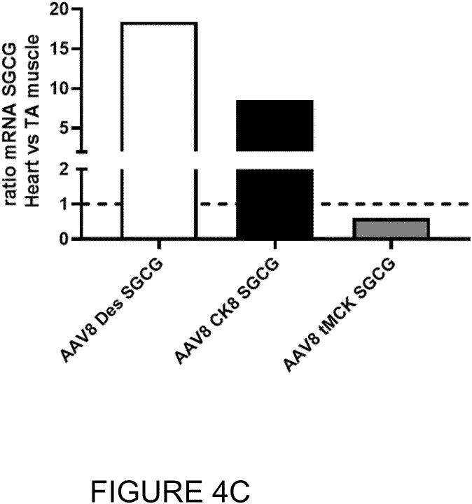

The level of SGCG mRNA in TA muscle was not clearly different between the 3 groups of mice. On the contrary, the activity of the tMCK promoter appeared much lower in the heart compared to the 2 other groups of mice, with a statistically significant difference at least with the CK8 promoter. As the number of tranduced cells in the liver is very high, the level of SGCG mRNA is also high (FIG. 4A).

The normalization of the mRNA SGCG abundance by the VGCN confirmed that the tMCK promoter activity is significantly different from both the Des and CK8 promoter activity (FIG. 4B).

Finally the SGCG mRNA ratio heart versus TA muscle obtained with the tMCK promoter (about 0.6) appeared to be more in adequation with the endogenous conditions (FIG. 4C), i.e. a higher expression in the TA muscle that in the heart.

These observations were confirmed by investigating SGCG protein expression in these different tissues:

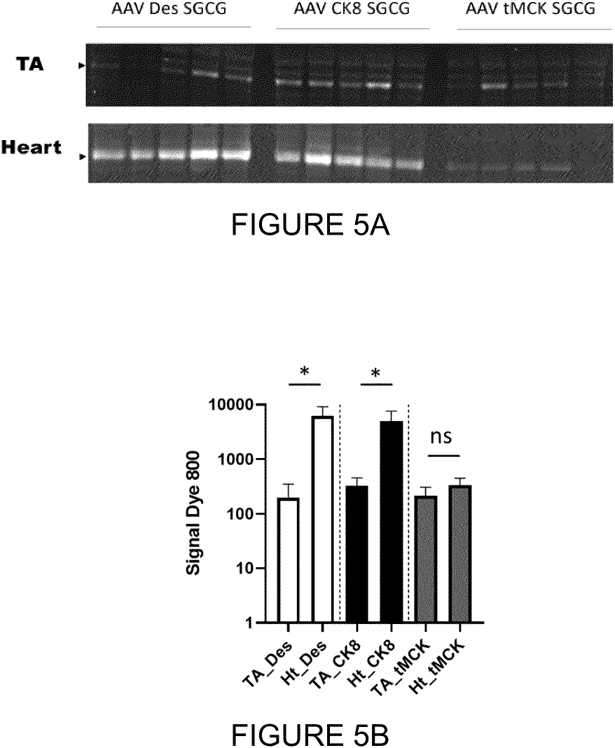

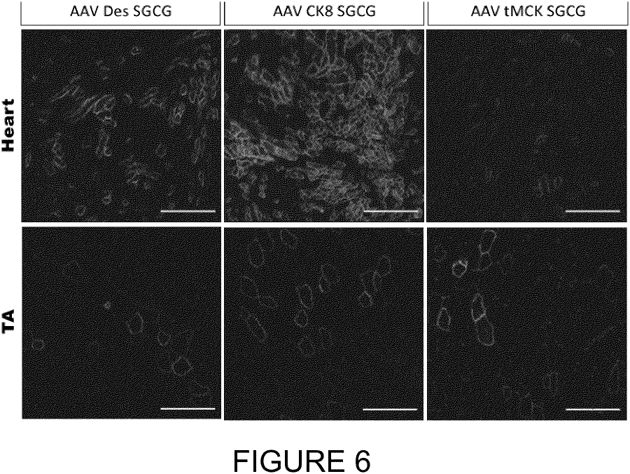

As revealed by FIG. 5, whereas the amount of transgene protein is significantly higher in heart than in TA muscle in the groups of mice injected with the AAV8-CK8-SGCG vector and with the AAV8-Des-SGCG vector, this is not the case for mice injected with the AAV8-tMCK-SGCG vector: the quantity of SGCCG is not significantly different between the heart and the TA muscle. Moreover, it is to be noted that the level of Sgcg protein in TA muscle is similar whatever the promoter used. Based on these results, the tMCK is confirmed to have an adequate expression profile, i.e.:
a high activity in the TA muscle similar to the desmin and CK8 promoters;
a lower activity in the heart than the desmin and CK8 promoters.
Direct observation on TA and cardiac tissues (FIG. 6) confirmed that the expression of SGCG in TA muscle was not clearly different between the 3 groups of mice. On the contrary, the heart from mice injected with the AAV8-tMCK-SGCG vector displayed fewer positive fibers compared with the 2 other groups of mice.

### IV/ Determination of Critical Amounts of SCGC in the Muscles and in the Heart

In order to determine the minimal therapeutically effective amount of SGCG in the muscles and the maximal not toxic amount of SGCG in the heart, further experiments were performed using the AAV8-Des-SGCG vector which has been shown above to lead to an adequate level of expression in the TA muscle but an excessive level of expression in the heart, possibly toxic.

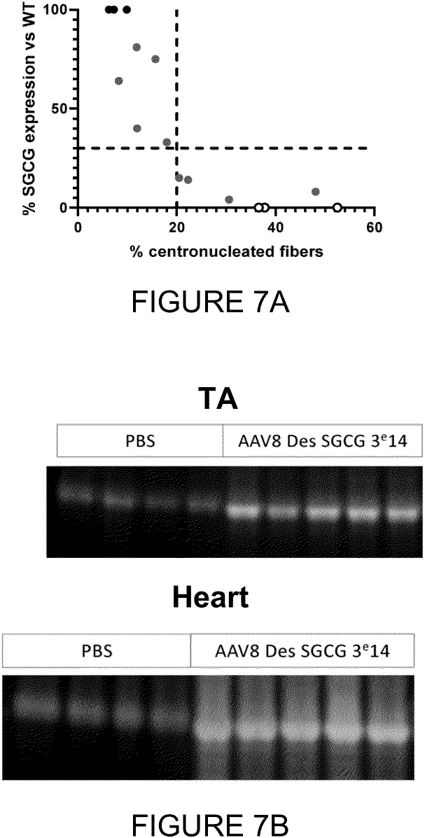

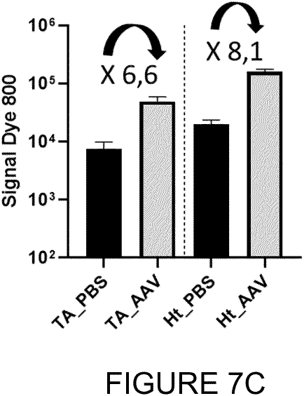

It can be concluded from FIG. 7A that in order to reach an acceptable centronucleation level (comparable or even slightly superior to the one observed in muscles of WT mice, i.e. up to 20%), the expression system should allow expressing at least 30% of the normal level of SGCG in the skeletal muscles.

On another hand, FIGS. 7B and 7C reveal that said system, potentially toxic in the heart, leads to a SGCG level in the heart 8 times greater than the one observed in the heart of WT mice.

### V/ Evaluation of Different Promoters in Rats

### V-1/Protein SGCG Expression Profile

The experiments disclosed above in mice were further performed in rats, adding as a new tested promoter, the MHCK7 promoter (AAV8-MHCK7-SGCG vector).

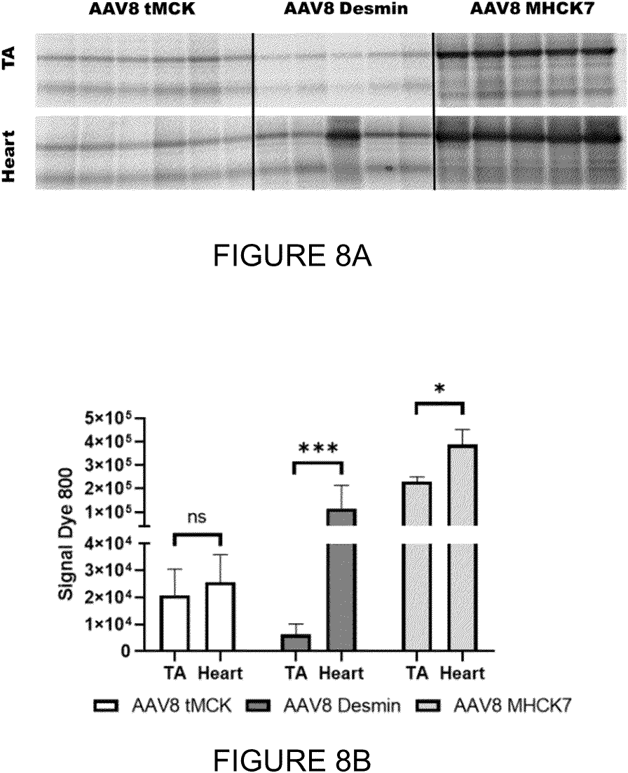

FIG. 8 reveals that in rats, the amount of transgene protein was significantly higher in heart than in TA muscles in the group of rats injected with the AAV8 Desmin-SGCG vector and with the AAV8 MHCK7-SGCG vector.

On the contrary, the transgene protein was equally expressed in the TA muscle and in the heart with the AAV8 tMCK-SGCG vector.

It is to be noted that the expression profile ratio obtained with the AAV8 Desmin-SGCG vector and the AAV8 tMCK-SGCG vector is similar in mice and in rats.

### V-2/ Impact on the Heart

The measurement of the transcript ratio Myh6/Myh7 is a good indicator to detect modification of the heart tissue that accompanies stress induced pathological conditions in heart (Scheuermann et al., EMBO J. 2013; 32(13): 1805-16).

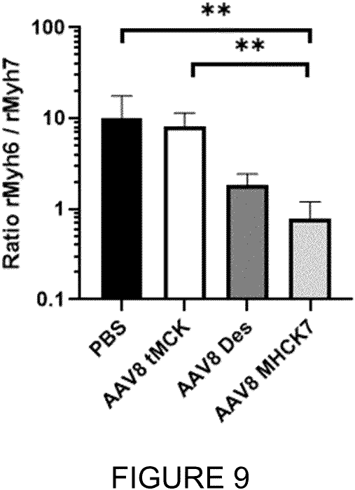

FIG. 9 shows that this ratio was not significantly modified in the group of rats injected with the vector AAV8 tMCK-SGCG (8.3) compared to the PBS control group (10.2). It further reveals that even if not statistically different, the ratio was strongly reduced in the heart of rats injected with the AAV8 Desmin-SGCG vector (1.8). Finally, the ratio was significantly lower in the heart of rats injected with AAV8 MHCK7-SGCG vector (0.8) in comparison with the PBS control and the AAV8-tMCK groups of rats.

Overall, the tMCK promoter is driving an equal expression between heart and skeletal muscle whereas with the two other promoters Desmin and MHCK7, SGCG is more expressed in the heart than in skeletal muscle as observed both in rat and mice. In addition, only the tMCK promoter conserves the correct ratio Myh6/Myh7 while this ratio is modified with the two other promoters, indicating cellular stress in the heart.

### CONCLUSIONS

As known in the art, the two most important organs that need to be targeted for the treatment of LGMD2C patients are the skeletal muscles and heart.

Based on the measurement of the endogenous SGCG protein in Wild Type mouse and macaca, it was concluded that the expression in the heart is preferably at the same level or even lower than in the skeletal muscles.

Regarding these different aspects, the AAV8-tMCK-SGCG vector was confirmed to be a very promising candidate. The level of expression is significantly reduced in heart compared to the 3 other promoters. Besides and in the TA muscle, the expression of the transgene is near to what obtained with the AAV8-Des-SGCG vector, a vector widely described as efficient to transduce the skeletal muscle and restore muscular activity (see e.g. Israeli et al., Mol Ther Methods Clin Dev. 2019; 13:494-502).

1-14. (canceled)

15. An expression system for systemic administration comprising a sequence encoding gamma-sarcoglycan (SGCG) placed under the control of a promoter allowing expression of SGCG in the skeletal muscles and in the heart, wherein the ratio between the SGCG expression in the skeletal muscles and the SGCG expression in the heart is superior or equal to 0.9.

16. The expression system according to claim 15, wherein the system is configured to express SGCG in the skeletal muscles in a quantity superior or equal to 0.3 times a quantity expressed endogenous.

17. The expression system according to claim 15, wherein the expression system is configured to express SGCG in the heart in a quantity inferior or equal to 8 times a quantity expressed endogenous.

18. The expression system according to claim 15, wherein the promoter is a tMCK promoter.

19. The expression system according to claim 18, wherein the tMCK promoter has a sequence as set forth in SEQ ID NO: 4.

20. The expression system according to claim 15, wherein the SGCG protein has the sequence as set forth in SEQ ID NO: 1 or SEQ ID NO: 2.

21. The expression system according to claim 15, wherein the sequence encoding the SGCG protein has the sequence SEQ ID NO: 3.

22. The expression system according to claim 15, wherein the expression system comprises SEQ ID NO: 5 or SEQ ID NO: 6.

23. The expression system according to claim 15, wherein the expression system comprises a viral vector.

24. The expression system according to claim 23, wherein the viral vector is an adeno-associated viral (AAV) vector.

25. The expression system according to claim 24, wherein the AAV vector is of serotype 8 or 9.

26. The expression system according to claim 25, wherein the expression system comprises an adeno-associated viral (AAV)2/8 or an AAV2/9 vector.

27. A pharmaceutical composition comprising the expression system according to claim 15.

28. The pharmaceutical composition according to claim 27, wherein the composition is a gene therapy composition.

29. A method of treating a pathology caused by a SGCG deficiency, comprising administering the pharmaceutical composition according to claim 27.

30. The method of claim 29, wherein the pathology caused by a SGCG deficiency is Limb-Girdle Muscular Dystrophy type C (LGMD2C or LGMD R5).

31. The method of claim 29, wherein the pharmaceutical composition is administered systemically.

32. The method of claim 31, wherein the pharmaceutical composition is administered by intravenous injection.
